(19) United States
(12) Patent Application Publication
(10) Pub. No.: US 2023/0190665 A1
(43) Pub. Date: Jun. 22, 2023

(54) THERAPEUTIC PROTEIN-LOADED NANOPARTICLE AND METHOD FOR PREPARING THE SAME

(71) Applicants: THE JOHNS HOPKINS UNIVERSITY, Baltimore, MD (US); SUN YAT-SEN UNIVERSITY, Guangdong (CN)

(72) Inventors: Hai-Quan Mao, Baltimore, MD (US); Jose Luis Santos, Rockville, MD (US); Zhiyu He, Baltimore, MD (US); Huahua Huang, Guangdong (CN); Lixin Liu, Guangdong (CN); Kam W. Leong, New York, NY (US); Yongming Chen, Guangdong (CN)

(21) Appl. No.: 16/606,456

(22) PCT Filed: Jan. 19, 2017
(86) PCT No.: PCT/US2017/014080

§ 371 Date: Oct. 18, 2019

(51) Int. Cl. A61K 9/51; A61K 38/28; A61P 3/10

(52) U.S. Cl. CPC A61K 9/5161; A61K 9/5192; A61K 38/28; A61K 9/5123; A61P 3/10

(57) ABSTRACT

The present invention belongs to the technical field of nanomedicine, and relates to a method for preparing a therapeutic protein-loaded nanoparticle, as well as a therapeutic protein-loaded nanoparticle, a suspension and a pharmaceutical composition comprising the nanoparticle, and a pharmaceutical preparation comprising the nanoparticle, the suspension or the pharmaceutical composition. The present invention further relates to a use of the nanoparticle in manufacture of a pharmaceutical composition, wherein the pharmaceutical composition is useful in prevention or treatment of a disease that can be prevented or treated by the therapeutic protein comprised in the nanoparticle.

### TECHNICAL FIELD

The present invention belongs to the technical field of nanomedicine, and relates to a method for preparing a therapeutic protein-loaded nanoparticle, as well as a therapeutic protein-loaded nanoparticle, a suspension and a pharmaceutical composition comprising the nanoparticle, and a pharmaceutical preparation comprising the nanoparticle, the suspension or the pharmaceutical composition. The present invention further relates to a use of the nanoparticle in manufacture of a pharmaceutical composition, wherein the pharmaceutical composition is useful in prevention or treatment of a disease that can be prevented or treated by the therapeutic protein comprised in the nanoparticle.

### BACKGROUND ART

Diabetes mellitus is a major disease following cardiovascular diseases and cancers that threatens human health. In the 1998 annual report of American Diabetes Association, it is pointed out there are about 135 million people with diabetes in the world, and the number of diabetic patients will rise to 300 million in 2025, in which the number will rise from 51 million to 72 million, an increase of 42%, in the developed countries; while in the developing countries, the number will jump from 84 million to 228 million, an increase of 170%. In the developed countries, there are nearly 16 million people with diabetes in the United States, accounting for about 5.9% of the total population of the United States, and about 100 billion US dollars is spend in the United States each year in prevention and treatment of diabetes. The prevalence of diabetes in China is not optimistic as well. A survey in 1998 shows that China has more than 20 million diabetic patients, and the incidence rate of diabetes in 25- to 64-year-old population is 2.5%. With the aging of population and changes in modern lifestyles in China, the prevention and treatment of diabetes has aroused widespread concern.

At present, insulin is one of the most effective drugs for treatment of diabetes, but it is usually used via subcutaneous injection. Long-term injection of insulin has many shortcomings, for example, patients get pains and fears; injection is not a convenient manner; content of insulin in local blood is too much, which stimulates the proliferation of smooth muscle cells, and converts glucose into lipid material on arterial wall; at insulin injection site, local precipitation of insulin would lead to local hypertrophy and fat precipitation; dependence on insulin; high cost; injection process may easily cause infection.

Oral taking of insulin is an administration route that is in most consistent with the manner of insulin physiological secretion, in which insulin directly enters into liver from intestine, thereby avoiding the occurrence of peripheral high concentration of insulin, and this is very meaningful for maintaining normal insulin sensitivity. However, insulin administration by oral route has the following problems: firstly, due to the acidic environment of stomach, insulin can easily be degraded in stomach; secondly, insulin can be degraded by enzyme and inactivated in digestive tract; finally, due to the high molecular weight and low lipid solubility of insulin, it has a low permeability in intestinal epithelial cells, leading to its low oral bioavailability. In recent years, nanocarriers are considered to have broad prospects in improvement of oral delivery of insulin.

Chitosan (CS) is produced by deacetylation of chitin, and is a natural polysaccharide with good physicochemical properties and widely used. It has characteristics of nontoxicity, biodegradability and biocompatibility. A large amount of active amino groups in chitosan molecules can be protonated in acidic medium to form polycationic electrolytes. Therefore, insulin-loaded nanoparticles can be prepared by cross-linking positively charged chitosan with polyanions.

In the prior art, the methods for preparing insulin-loaded nanoparticles using chitosan include: dropwise adding method and rapid dumping method. The nanoparticles prepared by the conventional methods are generally large in particle size and uneven in particle size distribution, and are unsatisfactory in controllability, stability and repeatability of the preparation process. Therefore, there is a need in the art for new methods for preparing insulin-loaded nanoparticles.

### Contents of the Invention

In the present invention, unless otherwise indicated, the scientific and technical terms used herein have the same meaning as commonly understood by one of ordinary skill in the art. Also, the laboratory procedures involved herein are conventional steps that are widely used in the relevant art. Meanwhile, for a better understanding of the present invention, definitions and explanations of related terms are provided below.

As used herein, the term “therapeutic protein” refers to a protein that is capable of being used for preventing or treating a disease.

As used herein, the term “nanoparticles” (NPs) refers to particles in nanoscale size (i.e., the diameter in the longest dimension of particle), for example, particles in size of not greater than 1,000 nm, not greater than 500 nm, not greater than 200 nm, or not greater than 100 nm.

As used herein, the term “particle” refers to a state of matter characterized by the presence of discrete particles, pellets, beads or agglomerates, regardless of their size, shape or morphology.

As used herein, the term “particle size” or “equivalent particle size” means that when a physical feature or physical behavior of a particle to be measured is most similar to a homogeneous sphere (or combination) of a certain diameter, the diameter (or combination) of the spheres is taken as the equivalent particle size (or particle size distribution) of the particle to be measured.

As used herein, the term “mean particle diameter” means that, for a actual particle population consisting of particles of different sizes and shapes, when it is compared to a hypothetical particle population consisting of homogeneous spherical particles, if their particle diameters are the same in full length, the diameter of the spherical particles is called the mean particle diameter of the actual particle population. The methods for measurement of mean particle diameter are known to those skilled in the art, for example, light scattering methods; and the mean diameter measurement instruments include, but are not limited to, Malvern particle size analyzer.

As used herein, the term “room temperature” refers to 25±5° C.

As used herein, the term “about” should be understood by those skilled in the art and will vary to some extent with the context in which it is used. The term “about” means not more than plus or minus 10% of a specific value or range, if the context in which the term is applied is not clear to a person skilled in the art.

As used herein, the term “preventing” refers to preventing or delaying the onset of a disease.

As used herein, the term “treating” refers to curing or at least partially arresting a disease, or alleviating a symptom of a disease.

The present inventors have obtained a method for preparing a therapeutic protein-loaded nanoparticle via in-depth research and creative labor. The method of the present invention is simple, mild and reproducible. In compared to current therapeutic protein-loaded nanoparticles, the nanoparticles prepared by the method of the present invention have smaller particle size, narrower particle size distribution and high encapsulation efficiency of protein, thereby providing the following invention:

In one aspect, the present application relates to a method for preparing a therapeutic protein-loaded nanoparticle, the method comprising the following steps:

Step 1: providing a chitosan solution, a polyanion solution, a therapeutic protein solution and water;

Step 2: allowing the chitosan solution, the polyanion solution, the therapeutic protein solution and the water separately to pass through a first channel, a second channel, a third channel and a fourth channel and to enter into a vortex mixing region, and mixing;

wherein, the chitosan solution, the polyanion solution, the therapeutic protein solution and the water flow at a uniform and constant flow rate in the channel; and the chitosan solution, the polyanion solution, the therapeutic protein solution and the water have a flow rate of 1-120 mL/min (e.g., 1 to 15 mL/min, 15 to 25 mL/min, 25 to 50 mL/min, 1 to 50 mL/min, 50 to 100 mL/min, or 100 to 120 mL/min).

In a preferred embodiment, the method is carried out in an apparatus comprising a first channel, a second channel, a third channel, a fourth channel and a vortex mixing region. In a preferred embodiment, the apparatus is a multi-inlet vortex mixer.

In a preferred embodiment, the therapeutic protein is insulin.

In a preferred embodiment, the polyanion is selected from sodium tripolyphosphate, alginic acid, heparin, hyaluronic acid, chondroitin sulfate, polyacrylic acid, polystyrene sulfonic acid; more preferably, the polyanion is sodium tripolyphosphate.

In a preferred embodiment, in Step 1, the chitosan solution, the therapeutic protein solution and the polyanion solution have a concentration ratio (mg/mL:mg/mL:mg/mL) of 1:0.1-0.7:0.2-0.5, for example 1:0.1-0.3:0.2-0.5, 1:0.3-0.5:0.3-0.5 or 1:0.35-0.70:0.2-0.35, for example 1:0.35-0.50:0.3-0.35, 1:0.35-0.70:0.2-0.35, 1:0.55-0.70:0.2-0.35, or 1:0.35-0.70:0.25-0.35.

In the present invention, the concentration of the chitosan solution refers to a mass concentration of chitosan contained in the chitosan solution; the concentration of the therapeutic protein solution refers to a mass concentration of therapeutic protein contained in the therapeutic protein solution; and the concentration of the polyanion solution refers to a mass concentration of polyanion contained in the polyanion solution.

In a preferred embodiment, in step 1, the concentration of the therapeutic protein solution is 0.1-0.7 mg/mL, for example 0.1-0.2 mg/mL, 0.2-0.3 mg/mL, 0.3-0.4 mg/mL, 0.4-0.5 mg/mL, 0.5-0.6 mg/mL, 0.6-0.7 mg/mL or 0.35-0.7 mg/mL, for example 0.1 mg/mL, 0.15 mg/mL, 0.2 mg/mL, 0.25 mg/mL, 0.3 mg/mL, 0.35 mg/mL, 0.4 mg/mL, 0.45 mg/mL, 0.5 mg/mL, 0.55 mg/mL, 0.6 mg/mL, 0.65 mg/mL or 0.7 mg/mL.

In a preferred embodiment, the therapeutic protein solution of Step 1 has a pH of 1.5-3.5, for example 1.5-2.0, 2.0-2.5, 2.0-3.0, 2.5-3.0 or 3.0-3.5, for example 1.5, 2.0, 2.5, 3.0, or 3.5.

In a preferred embodiment, the therapeutic protein solution of Step 1 further comprises hydrochloric acid.

In a preferred embodiment, the therapeutic protein solution of Step 1 is prepared by a method comprising steps of: dissolving a therapeutic protein in a hydrochloric acid solution having a pH of 1.5 to 3.5, for example a hydrochloric acid solution having a pH of 1.5-2.0, 2.0-2.5, 2.0-3.0, 2.5-3.0 or 3.0-3.5, for example a hydrochloric acid solution having a pH of 1.5, 2.0, 2.5, 3.0, or 3.5.

In a preferred embodiment, the therapeutic protein solution of Step 1 further comprises a therapeutic protein labeled with a fluorescent dye (for example FITC, Cy-3, Cy-5 and/or Cy-7).

In a preferred embodiment, the chitosan solution of Step 1 has a number average molecular weight of 10-500 kDa (for example 10-50 kDa, 50-90 kDa, 90-150 kDa, 150-190 kDa, 190-250 KDa, 250-350 KDa, or 350-500 KDa).

In a preferred embodiment, the chitosan solution of Step 1 has a pH of 5.0-6.0 (for example 5.0-5.3, 5.3-5.7 or 5.7-6.0, for example 5.0, 5.1, 5.2, 5.3, 5.4, 5.5, 5.6, 5.7, 5.8, 5.9 or 6.0).

In a preferred embodiment, the chitosan solution of Step 1 is prepared by a method comprising steps of: dissolving chitosan in an acetic acid solution having a concentration of 0.1% to 1% (for example 0.1% to 0.2%, 0.2% to 0.5%, 0.5% to 0.7% or 0.7% to 1.0%; for example 0.1%, 0.2%, 0.3%, 0.4%, 0.5%, 0.6%, 0.7%, 0.8%, 0.9% or 1.0%), and an alkali (for example, sodium hydroxide) is used to regulate the acetic acid solution to have a pH of 5.0-6.0 (for example 5.0-5.3, 5.3-5.7 or 5.7-6.0, for example 5.0, 5.1, 5.2, 5.3, 5.4, 5.5, 5.6, 5.7, 5.8, 5.9 or 6.0).

In a preferred embodiment, the chitosan solution of Step 1 further comprises chitosan labeled with a fluorescent dye (for example FITC, Cy-3, Cy-5 and/or Cy-7).

In a preferred embodiment, in the Step 1, the polyanionic solution has a concentration of 0.2-0.5 mg/mL, for example 0.2-0.3 mg/mL, 0.2-0.35 mg/mL, 0.35-0.4 mg/mL, 0.3-0.4 mg/mL or 0.4-0.5 mg/mL, for example 0.2 mg/mL, 0.25 mg/mL, 0.3 mg/mL, 0.35 mg/mL, 0.4 mg/mL, 0.45 mg/mL or 0.5 mg/mL.

In a preferred embodiment, the polyanion solution of Step 1 further comprises a buffering agent, for example 4-hydroxyethylpiperazineethanesulfonic acid (HEPES).

In a preferred embodiment, the pH of the polyanion solution of step 1 is 6.0-9.0, for example 6.0-7.0, 7.0-8.0 or 8.0-9.0.

In a preferred embodiment, the polyanion solution of Step 1 is prepared by a method comprising steps of: dissolving a polyanion in a HEPES buffer solution; more preferably, further comprising using an alkaline substance (for example, sodium hydroxide) to regulate the pH of the solution.

In a preferred embodiment, the water in Step 1 is double distilled water. Preferably, the mixing concentration is adjusted with the water.

In a preferred embodiment, a suspension is obtained in Step 2 of the method, and the suspension comprises a therapeutic protein-loaded nanoparticle.

In a preferred embodiment, the suspension obtained in Step 2 has a pH of 5.5-6.5 (for example 5.5-5.8, 5.8-6.0, 6.0-6.2 or 6.2-6.5, for example 5.5, 5.6, 5.7, 5.8, 5.9, 6.0, 6.1, 6.2, 6.3, 6.4 or 6.5).

In a preferred embodiment, the method further comprises Step 3: freeze drying the suspension.

In a preferred embodiment, the method further comprises: adding to the suspension a cryoprotectant prior to Step 3.

In a preferred embodiment, the cryoprotectant is selected from the group consisting of mannitol and xylitol.

In a preferred embodiment, the cryoprotectant is a combination of mannitol and xylitol.

In a preferred embodiment, the ratio of the mass of mannitol, the mass of xylitol to the volume of the suspension is 0.2-0.5 g: 0.5-1.5 g: 100 mL, for example 0.2-0.5 g: 0.5-1.0 g: 100 mL, 0.35-0.5 g: 0.5-1.0 g: 100 mL, 0.2-0.5 g: 1.0-1.5 g: 100 mL, or 0.2-0.5 g: 0.75-1.5 g: 100 mL.

In a preferred embodiment, the Step 2 is carried out in a multi-inlet vortex mixer.

In a preferred embodiment, the multi-inlet vortex mixer of the present invention comprises a first member located at the upper portion, a second member located at the middle portion and a third member located at the lower portion, wherein the first member, the second member and the third member are of cylinders with same diameter. The first member is provided with a plurality of channels, the second member is provided with a vortex mixing region and a plurality of diversion regions, and the third member is provided with a passageway. The channels of the first member are in fluid communication with the diversion regions of the second member. The diversion regions of the second member are in fluid communication with the vortex mixing region. The vortex mixing region of the second member is in fluid communication with the passageway of the third member. The first member, the second member and the third member can be hermetically connected using a threaded connection fitting.

In some embodiments, the first member is provided with a plurality of channels, and the channels have upper and lower ends separately located on the upper and lower surfaces of the first member. In some embodiments, the channels have cross-section in circle shape. In some embodiments, the channels are each connected to an external pipe through a connecting member.

In some embodiments, the upper surface of the second member is recessed with a plurality of diversion regions and a vortex mixing region. In some embodiments, the diversion regions are in fluid communication with the vortex mixing region through a slot provided on the upper surface of the second member. In some embodiments, the vortex mixing region of the second member is in fluid communication with the passageway of the third member through a passageway parallel to the axial direction of the second member.

In some embodiments, the cross-section of the vortex mixing region is circular and has a common center with the cross-section of the second member.

In some embodiments, the cross-section of the diversion regions is circular.

In some embodiments, the number of the diversion regions of the second member is the same as the number of the channels of the first member. In some embodiments, the diversion regions of the second member are each located right under the channels of the first member.

In some embodiments, the passageway of the third member has upper and lower ends, respectively, on the upper and lower surfaces of the third member. In some embodiments, the passageway of the third member is circular in cross-section. In some embodiments, the passageway of the third member is connected to an external pipe through a connecting member.

In some embodiments, the multi-inlet vortex mixer is made of a rigid material (for example, stainless steel).

An exemplary multi-inlet vortex mixer is shown in FIG. 1.

FIG. 1A shows a state in which the first member, the second member and the third member are assembled and connected to an external pipe, wherein the first member is located at the upper portion of the multi-inlet vortex mixer, the second member is located at the middle portion of the multi-inlet vortex mixer, the third member is located at the lower portion of the multi-inlet vortex mixer. The first member, the second member and the third member are hermetically connected by bolts. The four channels of the first member are respectively connected to external pipes through a connecting member. The passageway of the third member is also connected to an external pipe through a connecting member.

FIG. 1B-1 is a bottom view of the first member. As shown in the figure, the first member is provided with screw holes and channels; the upper and lower ends of the channels are respectively located on the upper surface and the lower surface of the first member.

FIG. 1B-2 is a top view of the second member. As shown in the figure, the second member is provided with screw holes; the upper surface of the second member is recessed with division regions and a vortex mixing region which have cross-sections in circular shape; the division regions are in fluid communication with the vortex mixing regions via a slot as set on the upper surface of the second member; the vortex mixing region has a passageway parallel to the axial direction of the second member.

FIG. 1B-3 is a top view of the third member. As shown in the figure, the third member is provided with a passageway channel and screw holes; and the upper and lower ends of the passageway are respectively located on the upper and lower surfaces of the third member.

In the multi-inlet vortex mixer shown in FIG. 1, the four diversion regions of the second member are each located directly below the four channels of the first member. Liquid can flow into the diversion regions of the second member through the channels of the first member, then enter the vortex mixing region, and then flow into the passageway of the third member through a passageway located in the center of the vortex mixing region.

In one aspect, the present application relates to a therapeutic protein-loaded nanoparticle, which comprises a therapeutic protein, chitosan and a polyanion, the nanoparticle having a particle size of from 30 to 240 nm (for example 30-60 nm, 60-90 nm, 90-120 nm, 120-150 nm, 150-180 nm, 180-210 nm or 210-240 nm, for example 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm or 240 nm), the nanoparticle having a particle diameter polydispersity index (PDI) of 0.13-0.19 (for example 0.13-0.15, 0.15-0.17 or 0.17-0.19, for example 0.13, 0.14, 0.15, 0.16, 0.17, 0.18 or 0.19), and the nanoparticle having an encapsulation efficiency of not less than 65% (for example not less than 65%, not less than 70%, not less than 75%, not less than 80%, not less than 85%, not less than 90% or not less than 95%).

In a preferred embodiment, the therapeutic protein is insulin.

In a preferred embodiment, the polyanion is selected from the group consisting of sodium tripolyphosphate, alginic acid, heparin, hyaluronic acid, chondroitin sulfate, polyacrylic acid, polystyrene sulfonic acid; more preferably, the polyanion is sodium tripolyphosphate.

In a preferred embodiment, the nanoparticle has a loading capacity of 10%-30%, for example 10%-15%, 15%-20%, 20%-25%, 25%-30%, 10%-20% or 20%-30%, for example 10%, 11%, 12%, 13%, 14%, 15%, 16%, 17%, 18%, 19%, 20%, 21%, 22%, 23%, 24%, 25%, 26%, 27%, 28%, 29% or 30%.

In a preferred embodiment, the nanoparticle has a Zeta potential of +5 mV to +15 my, for example +5 mV to +10 my or +10 mV to +15 my, for example +5 mV, +6 my, +7 mV, +8 my, +9 mV, +10 my, +11 mV, +12 my, +13 mV, +14 my or +15 my. Preferably, the Zeta potential is a Zeta potential of the nanoparticle existing in a suspension. Preferably, the suspension is prepared according to the method of the present invention.

In a preferred embodiment, in the nanoparticle, the mass ratio of chitosan to polyanion is 1:0.2-0.35, for example 1:0.2-0.25, 1:0.25-0.3 or 1:0.3-0.35, for example 1:0.2, 1:0.21, 1:0.22, 1:0.23, 1:0.24, 1:0.25, 1:0.26, 1:0.27, 1:0.28, 1:0.29, 1:3.0, 1:3.1, 1:3.2, 1:3.3, 1:3.4 or 1:3.5.

In a preferred embodiment, in the nanoparticle, the mass ratio of chitosan to the therapeutic protein is 1:0.1-0.7, for example 1:0.1-0.3, 1:0.2-0.35 or 1:0.35-0.7, for example 1:0.1, 1:0.15, 1:0.2, 1:0.25, 1:0.3, 1:0.35, 1:0.4, 1:0.45, 1:0.5, 1:0.55, 1:0.6, 1:0.65 or 1:0.7.

In a preferred embodiment, the nanoparticle exists in a suspension.

In a preferred embodiment, the nanoparticle is prepared according to the method of the present invention.

In one aspect, the present application relates to a suspension comprising a nanoparticle of the invention.

In a preferred embodiment, the suspension further comprises a cryoprotectant (for example mannitol and/or xylitol).

In a preferred embodiment, the suspension is prepared by the method of the present invention.

In one aspect, the present application relates to a pharmaceutical composition comprising a nanoparticle of the invention.

In a preferred embodiment, the pharmaceutical composition is used for prevention or treatment of a disease which can be prevented or treated by the therapeutic protein contained in the nanoparticle.

In a preferred embodiment, the therapeutic protein is insulin; the pharmaceutical composition is used for reducing blood glucose level in a subject.

In a preferred embodiment, the therapeutic protein is insulin; the pharmaceutical composition is used in prevention or treatment of hyperglycemia in a subject.

In a preferred embodiment, the hyperglycemia comprises stress-induced hyperglycemia; diabetes (including type 1 diabetes and type 2 diabetes) and impaired glucose tolerance.

In a preferred embodiment, the subject is a mammal, for example a bovine, an equine, a goat, a porcine, a canine, a feline, a rodent, a primate; for example, the subject is a human.

In one aspect, the present application relates to a pharmaceutical preparation comprising the nanoparticle, the suspension, or the pharmaceutical composition according to the present invention.

In a preferred embodiment, the pharmaceutical preparation further comprises a pharmaceutically acceptable excipient.

In a preferred embodiment, the pharmaceutical preparation is a lyophilized preparation.

In a preferred embodiment, the pharmaceutical preparation is a capsule.

In a preferred embodiment, the capsule has a capsule shell that is hydroxypropylmethyl cellulose ester capsule shell.

In a preferred embodiment, the pharmaceutical preparation is for preventing or treating a disease which can be prevented or treated by the therapeutic protein contained in the nanoparticle.

In a preferred embodiment, the therapeutic protein is insulin; the pharmaceutical composition is used for reducing blood glucose level in a subject.

In a preferred embodiment, the therapeutic protein is insulin; the pharmaceutical composition is used in prevention or treatment of hyperglycemia in a subject.

In a preferred embodiment, the hyperglycemia comprises stress-induced hyperglycemia; diabetes (including type 1 diabetes and type 2 diabetes) and impaired glucose tolerance.

In a preferred embodiment, the subject is a mammal, for example a bovine, an equine, a goat, a porcine, a canine, a feline, a rodent, a primate; for example, the subject is a human.

In one aspect, the present application relates to a use of the nanoparticle according to the present invention in manufacture of a pharmaceutical composition; the pharmaceutical composition is used in prevention or treatment of a disease which can be prevented or treated by the therapeutic protein contained in the nanoparticle.

In a preferred embodiment, the therapeutic protein is insulin, and the disease is hyperglycemia.

In a preferred embodiment, the hyperglycemia includes stress-induced hyperglycemia; diabetes (including type 1 diabetes and type 2 diabetes) and impaired glucose tolerance.

In a preferred embodiment, the subject is a mammal, for example a bovine, an equine, a goat, a porcine, a canine, a feline, a rodent, a primate; for example, the subject is a human.

In one aspect, the present application relates to a method for preventing or treating a disease, comprising administering the nanoparticle, the suspension, the pharmaceutical composition or the pharmaceutical preparation of the invention to a subject in need thereof, the disease being a disease which can be prevented or treated by the therapeutic protein contained in the nanoparticle, the suspension, the pharmaceutical composition or the pharmaceutical preparation.

In a preferred embodiment, the therapeutic protein is insulin, and the disease is hyperglycemia.

In a preferred embodiment, the hyperglycemia includes stress-induced hyperglycemia; diabetes (including type 1 diabetes and type 2 diabetes) and impaired glucose tolerance.

In a preferred embodiment, the subject is a mammal, for example a bovine, an equine, a goat, a porcine, a canine, a feline, a rodent, a primate; for example, the subject is a human.

### Beneficial Effects of the Invention

The method of the present invention can continuously and stably prepare the therapeutic protein-loaded nanoparticles in large-scale, and is superior to the existing preparation method in term of controllability, stability and repeatability of product.

The therapeutic protein-loaded nanoparticles of the present invention have one or more of the following beneficial effects:

(1) the nanoparticles of the present invention have a smaller particle size and/or a narrower particle size distribution;

(2) the nanoparticles of the invention have higher encapsulation efficiency and/or loading capacity;

(3) the surface of the nanoparticles of the present invention carries positive charges, which not only can provide static electricity stability for the nanoparticles, but also can enhance the interaction with negatively charged small intestinal mucous layer;

(4) the nanoparticles of the present invention do not undergo obvious dissociation or aggregation after freeze-drying, and the therapeutic protein in the nanoparticles do not show obvious leakage, and the properties of the nanoparticles are stable before and after lyophilization;

(5) the suspension of the nanoparticles of the present invention has good stability;

(6) the nanoparticles of the present invention are capable of reversibly opening the tight junctions of small intestinal epithelial cells and enhancing paracellular transport of the therapeutic protein;

(7) the nanoparticles of the present invention can effectively control the blood sugar level by oral administration.

Embodiments of the present invention will now be described in detail with reference to the accompanying drawings and examples, but it will be understood by those skilled in the art that the following drawings and examples are merely illustrative of the present invention and are not to be construed as limiting the scope of the present invention. The various objects and advantages of the present invention will become apparent to those skilled in the art from the following detailed description of the drawings and preferred embodiments.

### BRIEF DESCRIPTION OF THE DRAWINGS

FIG. 1 illustrates schematically a multi-inlet vortex mixer for preparing the nanoparticle of the present invention. FIG. 1A shows a state in which the first member, the second member and the third member are assembled and connected to external pipes; FIG. 1B-1 is a bottom view of the first member; FIG. 1B-2 is a top view of the second part; and FIG. 1B-3 is a top view of the third part.

FIG. 2 shows an apparatus for preparing nanoparticle in Example 1, in which FIG. 2A shows syringes, high pressure pumps, plastic pipes and the multi-inlet vortex mixer, and FIG. 2B is an enlarged view of the multi-inlet vortex mixer connected to plastic pipes.

FIG. 3 shows the results of particle size measurement and morphological characterization of blank nanoparticles, Nanoparticles 1 and Nanoparticles 2 as prepared in Example 1.

FIGS. 3A-C shows the results of blank nanoparticles, Nanoparticles 1 and Nanoparticles 2 as tested by using a Malvern particle size analyzer. The blank nanoparticles, Nanoparticles 1 and Nanoparticles 2 had average particle sizes of 37.7 nm, 45.4 nm and 117.7 nm, respectively. The Nanoparticles 1 and Nanoparticles 2 had PDIs of 0.139 and 0.146, respectively. The results showed that the insulin-loaded nanoparticles prepared by the method of the present invention had small particle size and narrow particle size distribution, and their particle size was similar to that of the insulin-free nanoparticles as prepared under the same conditions.

FIG. 3D-I showed the TEM photos of blank nanoparticle, Nanoparticles 1 and Nanoparticles 2, in which FIGS. 3D and 3G showed the photographs of the blank nanoparticles, FIGS. 3E and 3H showed the photographs of Nanoparticles 1, and FIGS. 3F and 3I showed the photographs of Nanoparticles 2. As shown, the nanoparticles are approximately spherical in shape and uniform in particle size distribution.

FIG. 4 shows the particle size and polydispersity index of nanoparticles prepared at different flow rates. As shown, the nanoparticles prepared at a flow rate of 1 mL/min to 50 mL/min had particle size of not more than 120 nm, and PDI of not more than 0.2. When the flow rate was 1 mL/min to 25 mL/min, the nanoparticles had particle size of 120 nm to 45 nm, and PDI of 0.172-0.139; when the flow rate was 25 mL/min to 50 mL/min, the particle diameter was 45 nm to 55 nm, and the PDI was 0.139-0.190. The above results show that insulin-loaded nanoparticles with small particle size and narrow particle size distribution can be prepared by the method of the invention, and the size of nanoparticles can be regulated by adjusting the flow rate.

FIG. 5 shows the release of insulin from Nanoparticles 1 in PBS solution at pH 7.4 in Example 5, and the stability of the released insulin. FIG. 5A shows the cumulative release profile of insulin. As shown, 40% of insulin was released within 4 hours, which indicated a relatively rapid insulin release rate. FIG. 5B shows the results of circular dichroism spectra, and it can be seen from the figure that the conformation of the insulin released from the nanoparticles did not change in comparison with insulin standard sample, which indicates that the insulin in the nanoparticles was stable in term of structure.

FIG. 6 shows curves of trans-epithelial electrical resistance (TEER) versus time for Caco-2 monolayer cells under the action of insulin-loaded nanoparticles (Nanoparticles 1 or Nanoparticles 2) or free insulin in a solution of Example 7. In the figure, the abscissa is time and the ordinate shows the change of TEER. As shown in the figure, Nanoparticles 1 and Nanoparticles 2 resulted in rapid decreases of the TEER of Caco-2 monolayer cells to 50% and 54% of the initial values, respectively, within 2 hours after the start of the experiment; whereas the free insulin reduced the TEER of Caco-2 monolayer cells to about 85% of the initial value. Thus, in comparison with the free insulin, the insulin-loaded nanoparticles caused significantly faster decrease of the TEER of Caco-2 monolayer cells, indicating that the insulin-loaded nanoparticles were more likely able to open the tight junctions of the cells. After 2 hours after the start of the experiment, the nanoparticles or the insulin solution were removed and the TEER of cells of each experimental group was slowly picked up.

FIG. 7 is a curve of accumulative amount of transported insulin versus time in Example 7. As shown in the figure, in comparison with the free insulin, the insulin as loaded by the Nanoparticles 1 or Nanoparticles 2 showed significantly higher amount of transport.

FIG. 8 shows the effect of Nanoparticles 1 on stained Caco-2 monolayer cells in Example 7. The figure shows the morphologies of the cells as observed under a confocal microscopy before the action of the nanoparticles (FIG. 8A), under the action of the nanoparticles (FIG. 8B) and after the nanoparticles were removed (FIG. 8C-F). It can be observed that tight junction proteins showed a continuous loop along the cell boundary before the action of the nanoparticles. After two hours of the action of the nanoparticles, the tight junction proteins became blurred, and the loop along the cell boundary became discontinuous, indicating that the tight junctions of cells were opened. When the nanoparticles were removed, the tight junction proteins became clear and the morphologies of the proteins were gradually recovered. The above results indicated that, the insulin-loaded nanoparticles of the invention are capable of reversibly opening the tight junctions of cells.

FIG. 9 shows the effect of the nanoparticles labeled with both FITC and Cy-5 on insulin transport as observed under a confocal microscopy in Example 8. In the figure, Columns 1-3 show the results of characterization of Nanoparticles 3, Columns 4-6 show the results of the characterization of Nanoparticles 4, and Column 7 shows the results of characterization of the control group (free insulin). Nanoparticles 3 and Nanoparticles 4 had strong fluorescence signals of Cy-5 at depths of 6 μm and 12 μm after incubation for 2 hours, indicating that the insulin released from Nanoparticles 3 and Nanoparticles 4 was transported in Caco-2 monolayer cells. However, the control group had only weak Cy-5 fluorescence signals at depths of 6 μm and 12 μm. These results indicate that the nanoparticles of the present invention can enhance the insulin transport by cells.

FIG. 10 shows curves of blood glucose level versus time in each of the groups of rats in Example 9. Group 1: intragastrically administrated with Nanoparticles 1 at a dose of 60 IU/kg; Group 2: intragastrically administrated with Nanoparticles 1 at a dose of 120 IU/kg; Group 3: subcutaneously injected with a free insulin solution at a dose of 10 IU/kg; Group 4: intragastrically administrated with a free insulin solution at a dose of 60 IU/kg; Group 5: orally administrated with blank nanoparticles; Group 6: orally administrated with deionized water. As shown in the figure, the rats of Group 1 showed a blood glucose decreased by 51% within 8 hours after being intragastrically administrated with the nanoparticles at a dose of 60 IU/kg. The rats of Group 2 showed a blood glucose decreased by 59% within 8 hours after being intragastrically administrated with the nanoparticles at a dose of 120 IU/kg. The rats of Group 3 showed a sharp drop in blood glucose to 20% of the basal level within 1 hour after being subcutaneously injected with the free insulin solution at a dose of 10 IU/kg, and this was further maintained for 4 hours. The rats of Group 4 showed no significant drop in blood glucose level after being orally administrated with the free insulin solution, while the rats of Group 5 and Group 6 showed similar results of blood glucose level. After 8 hours later, the rats were not fasted, and their blood glucose levels were picked up. On the next day, the same experiment was repeated, and similar results of blood glucose levels were observed. The above results show that the insulin-loaded nanoparticles of the present invention can effectively reduce blood glucose level by oral administration, without causing a sharp decline in blood glucose level.

FIG. 11 shows the results of intraperitoneal glucose tolerance test in Example 10. As shown in the figure, after being injected with a glucose solution, the mice administrated with the nanoparticles (Nanoparticles 1 or Nanoparticles 2) as prepared by the method of the present invention did not show an increase of blood glucose level; the mice administrated with the nanoparticles (Nanoparticles 3) as prepared by dropwise adding method showed an increase of blood glucose level of about 2 mM; while the mice administrated with free insulin showed an increase of blood glucose level of about 8 mM. The above results show that the insulin-loaded nanoparticles as prepared by the method of the present invention can effectively control the blood glucose level.

FIG. 12 shows the distribution of insulin-loaded nanoparticles in rats in Example 11. FIG. 12A shows the pictures of 1 hour, 2 hours, 4 hours and 6 hours after intragastrical administration of the suspension; and FIG. 12B shows the pictures of 1 hour, 2 hours, 4 hours, and 6 hours after the intragastrical administration of the capsules. As shown in the figure, when 6 hours after the rats were administrated with the suspension, there was still a lot of insulin in stomachs of the rats, while some of insulin was located in liver, kidneys and intestines. When 6 hours after the rats were administrated with the capsules, most of insulin was located in intestines, while some of insulin was located in the liver and kidneys. The results show that encapsulating the insulin-loaded nanoparticles in the capsules can decrease the release of insulin in stomach, so that insulin is released more in small intestine, thereby enhancing the absorption of insulin on surface of small intestine and further increasing bioavailability thereof.

FIG. 13 shows concentration-time curves of serum insulin concentration in rats in Example 12. Group I: intragastrically administrated with HPMCP capsules of Nanoparticles 1 (60 IU/kg); Group II: intragastrically administrated with HPMCP capsules of insulin powder (60 IU/kg); and Group III: subcutaneously injected with a free insulin solution (5 IU/kg). The concentration-time curve of Group I shows that after 3 hours from the administration, serum insulin began to be detected, and reached to a peak value after 5 hours (Cₘₐₓ=45.4 mIU/L). No insulin was detected in serum in the rats of Group II. The concentration-time curve of Group III shows that the insulin concentration in serum increased sharply after administration (which might cause a sharp drop of blood glucose level), and reached to a peak value (Cₘₐₓ=73.5 mIU/L) after 1 hour from the administration. The relative bioavailability of the capsule comprising the insulin-loaded nanoparticles was calculated to be 10%.

FIG. 14 shows the results of biosafety evaluation of the insulin-loaded nanoparticles in Example 13. As shown in the figure, in comparison with the rats administrated with free insulin and the rats of the control group (not administered), the rats administrated with the insulin-loaded nanoparticles showed no significant difference in various indexes. The results show that the insulin-loaded nanoparticles of the present invention have good biosafety.

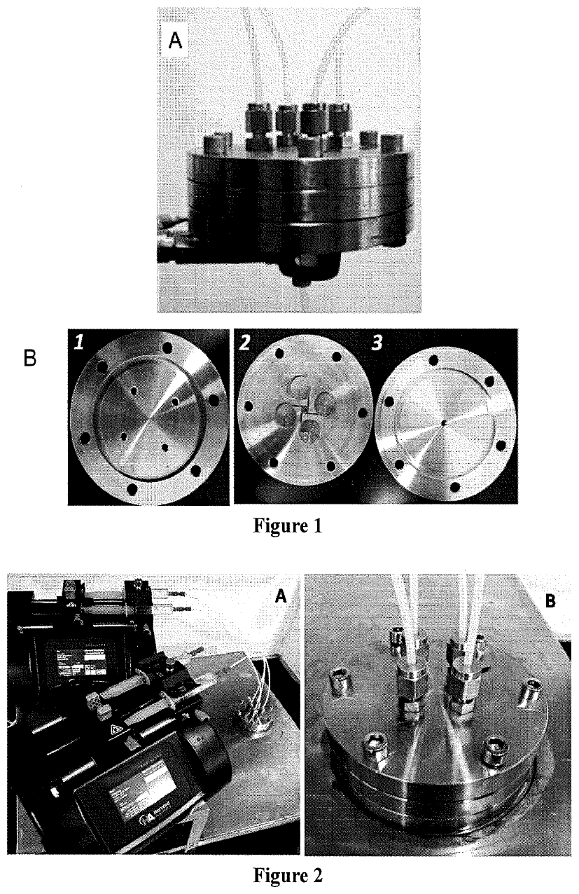

### SPECIFIC MODELS FOR CARRYING OUT THE INVENTION

Embodiments of the present invention will now be described in detail with reference to the following examples, but it will be understood by those skilled in the art that the following examples are illustrative only and are not intended to limit the scope of the invention. Specific conditions that are not given in the examples would be carried out in accordance with conventional conditions or the manufacturer's recommended conditions. When manufactures of the used reagents or instruments are not marked, they are all conventional products commercially available.

### Example 1: Preparation of Insulin-Loaded Nanoparticles

1. Preparation Process:

(1) Insulin was dissolved in a hydrochloric acid solution of pH 2.8 to give an insulin solution with a concentration of 0.5 mg/mL.

(2) Chitosan (90 KDa, 85% deacetylated) was dissolved in 0.2% acetic acid solution to give 1 mg/mL chitosan solution, and its pH value was adjusted to 5.3 by NaOH solution.

(3) Sodium tripolyphosphate was dissolved in 0.025 M HEPES buffer to give 0.2 mg/mL sodium tripolyphosphate solution.

(4) The chitosan solution, the sodium tripolyphosphate solution, the insulin solution and double distilled water were respectively loaded into four syringes, and the four syringes were respectively placed on high-pressure pumps. The injection holes of the syringes were respectively hermetically connected with ends of plastic pipes 1-4, while the other ends of the plastic pipes were separately hermetically connected with the four channels of the first member of the multi-inlet vortex mixer through the connecting member. The first member, the second member and the third member of the multi-inlet vortex mixer were hermetically connected by bolts, and the passageway of the third member was hermetically connected to one end of the plastic pipe 5 through a connecting member, while the other end of the plastic pipe 5 was connected to the collecting container. FIG. 2 shows the apparatus for preparing the nanoparticles of this Example, in which FIG. 2A shows syringes, high pressure pumps, plastic pipes, and the multi-inlet vortex mixer, and FIG. 2B shows an enlarged view of the multi-inlet vortex mixer connected with plastic pipes.

(5) The high-pressure pump was turned on so that the chitosan solution, the sodium tripolyphosphate solution, the insulin solution and the double distilled water were simultaneously introduced into the multi-inlet vortex mixer through the plastic pipes 1-4 at the same flow rate of 25 mL/min, and mixed in the vortex mixing region of the second member to obtain a suspension of insulin-loaded nanoparticles (Nanoparticles 1), which was flowed through a plastic pipe 5 into the collection container.

(6) 5 mL of the suspension was taken, added with cryoprotectant (0.5% (g/mL) mannitol and 1% (g/mL) xylitol), frozen at −80° C. for 72 hours, and lyophilized in a lyophilizer to obtain a lyophilized preparation (white solid) according to a scheduled lyophilization procedure.

2. According to the operation and parameters of steps (1)-(6), the insulin solution was replaced with a hydrochloric acid aqueous solution of pH2.8 to prepare blank nanoparticles.

3. According to the steps (1)-(6), the flow rate of liquid in the channels was 1 mL/min, and the flow rates of the four liquids were the same, and other conditions were not changed, so as to prepare the insulin-loaded nanoparticles (Nanoparticles 2) and a lyophilized preparation.

4. The preparation was carried out according to the steps (1)-(6), in which the flow rate of liquid in channel was 5 mL/min, 10 mL/min, 15 mL/min, 20 mL/min, 30 mL/min, 35 mL/min, 40 mL/min, 45 mL/min or 50 mL/min, and the flow rates of the four liquids were the same, and the other conditions were kept unchanged to prepare the suspension of the insulin-loaded nanoparticles.

5. The preparation was carried out according to the steps (1)-(6), in which sodium tripolyphosphate solutions at different concentrations (0.2 mg/mL, 0.25 mg/mL or 0.35 mg/mL) and insulin solutions at different concentrations (0.35 mg/mL, 0.5 mg/mL or 0.7 mg/mL) were used, and the flow rate of each liquid was always kept at 25 mL/min.

6. The preparation was carried out according to the steps (1)-(6), in which the used sodium tripolyphosphate solutions had a concentration of 0.2 mg/mL and different pH values, while the concentrations, pH values and flow rates of the other solutions were the same for preparing Nanoparticles 1.

7. The insulin solution, chitosan solution and sodium tripolyphosphate solution in steps (1)-(3) were used, and dropwise adding method and rapid dumping method were used to prepare insulin-loaded nanoparticles useful in comparative experiments.

Dropwise adding method: under stirring, the sodium tripolyphosphate solution and insulin solution as well as water were simultaneously added dropwise to the chitosan solution at a dropping rate of 1 drop/s (about 20 μL/s), and the final volume ratio of these 3 solutions and water was 1:1:1:1.

Rapid dumping method: under stirring, the sodium tripolyphosphate solution and insulin solution as well as water were simultaneously poured into the chitosan solution, the volume ratio of these 3 solutions and water was 1:1:1:1.

8. Nanoparticles 3 for comparative experiments were prepared by using chitosan solution (pH=5.2, 2 mg/mL), insulin solution (pH=7.0, 1 mg/mL) and sodium tripolyphosphate solution (pH=9.0, 0.5 mg/mL) according to the above dropwise adding method.

### Example 2. Measurement of Particle Size, Measurement of Electric Potential and Characterization of Morphology

1. Measurement of Particle Size:

The particle size and polydispersity index (PDI) of the nanoparticles in the suspensions were determined using a Malvern particle size analyzer (with a dynamic light scattering detector).

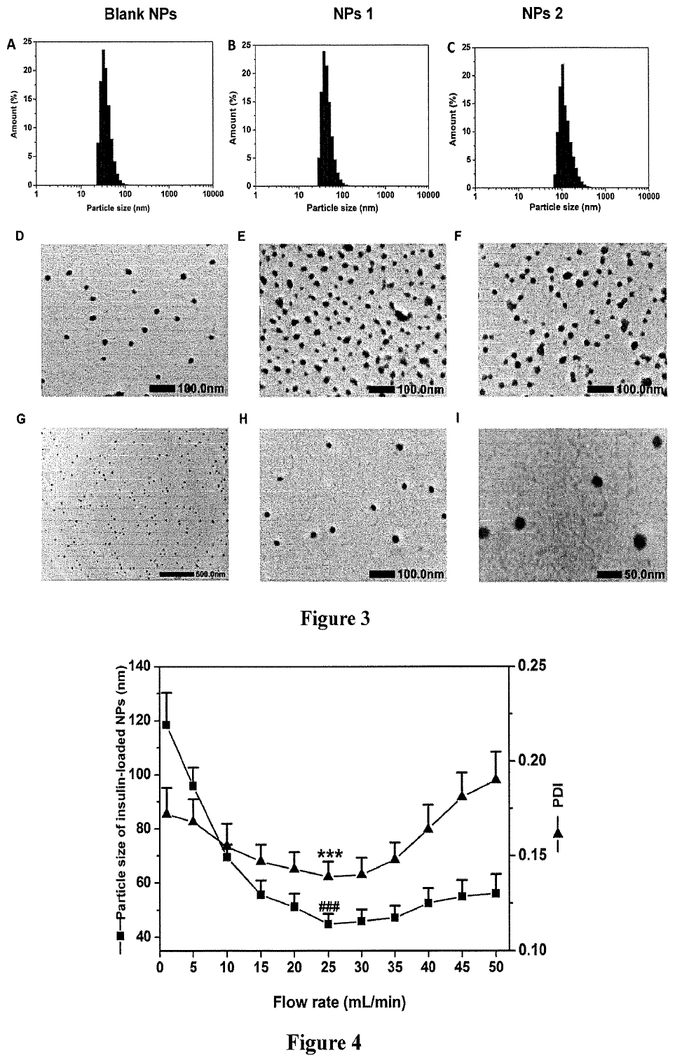

FIGS. 3A, B, C separately show the results of blank nanoparticles, Nanoparticles 1 and Nanoparticles 2 as measured by using a Malvern particle size analyzer. The blank nanoparticles, Nanoparticles 1 and Nanoparticles 2 had average particle sizes of 37.7 nm, 45.4 nm and 117.7 nm, respectively. The Nanoparticles 1 and Nanoparticles 2 had PDIs of 0.139 and 0.146, respectively. The results showed that the insulin-loaded nanoparticles prepared by the method of the present invention had small particle size and narrow particle size distribution, and their particle size was similar to that of the insulin-free nanoparticles as prepared under the same conditions.

FIG. 4 shows the average particle sizes and PDIs of nanoparticles prepared at different flow rates. As shown, the nanoparticles prepared at a flow rate of 1 mL/min to 50 mL/min had particle size of not more than 120 nm, and PDI of not more than 0.2. When the flow rate was 1 mL/min to 25 mL/min, the nanoparticles had particle size of 120 nm to 45 nm, and PDI of 0.172-0.139; when the flow rate was 25 mL/min to 50 mL/min, the particle diameter was 45 nm to 55 nm, and the PDI was 0.139-0.190. The above results show that insulin-loaded nanoparticles with small particle size and narrow particle size distribution can be prepared by the method of the invention, and the size of nanoparticles can be regulated by adjusting the flow rate.

Table 1 shows the particle sizes of the nanoparticles as prepared under conditions using sodium tripolyphosphate solutions and insulin solution with different concentrations at a liquid flow rate of 25 mL/min.

TABLE 1

| Concentration ratio of chitosan solution:sodium | Average |
|---|---|
| tripolyphosphate solution:insulin solution | particle |
| (mg/mL:mg/mL:mg/mL) | size |
| 1:0.35:0.35 | 41 ± 3.4 nm |
| 1:0.25:0.35 | 50 ± 3.7 nm |
| 1:0.2:0.35 | 55 ± 4.2 nm |
| l:0.2:0.5 | 56 ± 7.1 nm |
| l:0.2:0.7 | 57 ± 8.3 nm |

As shown in Table 1, the particle size of the nanoparticles could be regulated by adjusting the concentrations of the raw material solutions.

Table 2 shows the average particle sizes and the PDIs of the nanoparticles as prepared by the method of the present invention, the dropwise adding method and the rapid dumping method.

TABLE 2

| | Average particle size | PDI |
|---|---|---|
| The method of the present invention | 45 ± 4.1 nm | 0.127 |
| Dropwise adding method | 92 ± 8.4 nm | 0.16 |
| Rapid dumping method | 105 ± 9.1 nm | 0.20 |

These results demonstrate that the method of the present invention is capable of preparing nanoparticles having smaller particle size and narrower particle size distribution than the conventional methods for preparing insulin-loaded nanoparticles.

2. Measurement of Potential:

The zeta potential of Nanoparticles 1 was measured by Malvern particle size analyzer (with Zeta potential test function), which was +9.4 mV, indicating that positive charges were carried on the surface of the nanoparticles, the nanoparticles could be electrostatically stabilized, and the interaction with the negatively charged intestinal mucous layer could be enhanced, thereby facilitating the absorption of nanoparticles through intestinal epithelium.

3. Characterization of Morphology:

FIG. 3D-I showed the TEM photos of blank nanoparticles, Nanoparticles 1 and Nanoparticles 2, in which FIGS. 3D and 3G showed the photographs of the blank nanoparticles, FIGS. 3E and 3H showed the photographs of Nanoparticles 1, and FIGS. 3F and 3I showed the photographs of Nanoparticles 2. As shown, the nanoparticles were approximately spherical in shape and uniform in particle size distribution. The average particle sizes of the nanoparticles were statistically consistent with those obtained using the particle size analyzer.

### Example 3: Calculation of Encapsulation Efficiency and Loading Capacity

The suspension containing Nanoparticles 1 was ultrafiltered at 3000 rpm for 20 min, then the ultrafiltrate was measured for UV absorbance and compared with standard insulin samples, and the encapsulation efficiency and loading capacity of the nanoparticles were calculated according to the following formula:

Encapsulation efficiency=(total drug amount−free drug amount)/total drug amount×100%;

Loading capacity=total drug amount in nanoparticles/total amount of nanoparticles×100%.

According to calculation, Nanoparticles 1 had an encapsulation efficiency of 91% and a loading capacity of 27.5%.

The preparation was carried out using 3 sodium tripolyphosphate solutions with different pH values, the obtained suspensions had pH of 6.0, 6.2 and 6.5, respectively, and the nanoparticles in these suspensions had encapsulation efficiencies of 65%, 80% and 90%, respectively.

The nanoparticles were prepared by the method of the present invention, the dropwise adding method and the rapid dumping method under condition of keeping the raw material solution unchanged, and their encapsulation rates were shown in Table 3.

TABLE 3

| | Encapsulation efficiency |
|---|---|
| The method of the present invention | 91% |
| Dropwise adding method | 62% |
| Rapid dumping method | 42% |

The results show that the insulin-loaded nanoparticles prepared by the method of the present invention have high encapsulation efficiency, and the encapsulation efficiency of the nanoparticles can be regulated by adjusting pH of raw material solutions.

### Example 4: Characterization of Lyophilized Insulin-Loaded Nanoparticles

The lyophilized preparation of Nanoparticles 1 was hydrated to give a suspension. The nanoparticles therein were tested and compared with the nanoparticles in the suspension before lyophilization. The results are shown in Table 4.

TABLE 4

| | Before lyophilization | After lyophilization |
|---|---|---|
| Average particle size(nm) | 46.2 ± 2.7 | 45.3 ± 3.7 |
| Zeta potential (mv) | 9.4 ± 1.2 | 9.1 ± 1.7 |
| PDI | 0.15 ± 0.02 | 0.15 ± 0.03 |
| Encapsulation effiency | 91% ± 1.7% | 90.2% ± 2.4% |
| Loading capacity | 27.5 ± 0.4% | 27.3 ± 0.5% |
| pH of suspension | 6.5 | 6.5 |

It can be seen from the results of the Table that the particle sizes, particle size distributions, zeta potentials, encapsulation efficiencies and loading capacities of the nanoparticles did not change significantly before and after lyophilization; and the pH of suspensions did not change significantly before and after lyophilization as well. The results show that there was no obvious dissociation or aggregation of nanoparticles after lyophilization, and there was no obvious leakage of insulin from the nanoparticles. The properties of the nanoparticles remained stable before and after lyophilization.

### Example 5: Experiments for pH Stability and In Vitro Release of Insulin-Loaded Nanoparticles

1. PBS solution of pH 6.6 was used to stimulate the environment of duodenum and jejunum for testing the particle size and insulin release of Nanoparticles 1. After staying in the environment of pH 6.6 for 1 hour, Nanoparticles 1 had an average particle size of 53 nm and an insulin release of about 3%. The results showed that the nanoparticles of the present invention were stable in the pH 6.6 environment without significant degradation or aggregation and no significant leakage of insulin.

2. PBS solution of pH 7.4 was used to simulate the intercellular humoral environment for testing the insulin release of Nanoparticles 1. The nanoparticles were put into PBS solution of pH 7.4, stirred at 100 rpm at room temperature, and samples were taken out after certain time intervals, ultra-filtrated, and the supernatant was subjected to BCA protein analysis. The released insulin was tested using circular dichroism spectrum analysis, and the stability of the released insulin was evaluated by comparison with the spectra of insulin standard.

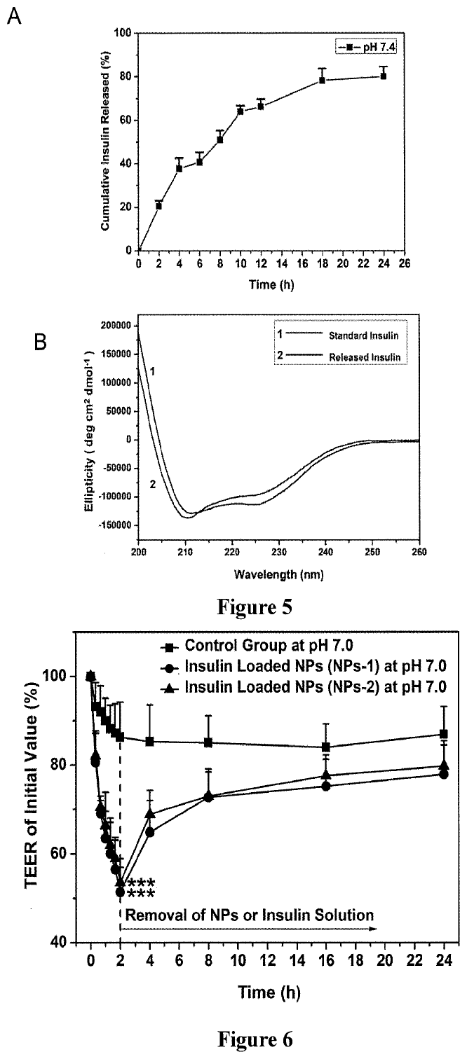

FIG. 5A shows the accumulative release profile of insulin of Nanoparticles 1 in PBS of pH 7.4. As shown in the figure, 40% of insulin was released within 4 hours, indicating a rapid insulin release rate. It can be seen from the results of circular dichroism spectrum analysis as shown in FIG. 5B that the conformation of insulin released from the nanoparticles did not change significantly in comparison with insulin standard, indicating that the structure of insulin in the nanoparticles was stable.

### Example 6: Stability Test of Insulin-Loaded Nanoparticles

Nanoparticles 1 obtained in Example 1 were allowed to stand at room temperature for one week, then the particle size and encapsulation efficiency of the nanoparticles were measured and compared with those before standing, and the results are shown in Table 5.

TABLE 5

| | Before standing | One week after standing |
|---|---|---|
| Average particle size(nm) | 45.4 | 48 |
| Encapsulation efficiency | 91% | 87% |

The results show that the particle size of the nanoparticles in the suspension was unchanged after the suspension stood for one week, indicating that aggregation or dissociation of the nanoparticles was not obvious; and the encapsulation efficiency changed little, indicating that the leakage of insulin from the nanoparticles was not obvious.

### Example 7: Effects of Insulin-Loaded Nanoparticles on Paracellular Transport

Caco-2 cells are human cloning colonic adenocarcinoma cells which are similar to differentiated small intestinal epithelial cells in structure and function and can be used for experiment of simulating in vivo intestinal transport. In the present invention, the Transwell test of Caco-2 monolayer cells was used for investigation of transcellular transport of insulin-loaded nanoparticles. When tight junctions of cells were opened, trans-epithelial electrical resistance (TEER) of monolayer cells would be reduced. Therefore, by measuring TEER of Caco-2 monolayer cells, the opening degree of tight junctions of cells could be evaluated, and effects of insulin-loaded nanoparticles on paracellular transport of intestinal epithelial cells could be studied. Meanwhile, the tight junction proteins could be fluorescent stained to observe the changes of tight junctions.

Cell culture: Caco-2 cells were incubated in a 12-well polycarbonate membrane chamber (diameter: 12 mm, growth area: 1.12 cm², membrane pore size: 0.4 μm), and were used in the test after incubation for 16-21 days (stable TEER was 700-800 f/xcm²). Samples to be tested: a suspension of Nanoparticles 1 (insulin concentration 0.2 mg/mL, 0.5 mL, pH 7.0); a suspension of Nanoparticles 2 (0.2 mg/mL, 0.5 mL, pH 7.0). Blank control: a free insulin solution (0.2 mg/mL, pH 7.0).

1. Measurement of TEER

The samples to be tested or the blank control were added to an incubation chamber and incubated at 37° C. The TEER of Caco-2 monolayer cells under action of insulin-loaded nanoparticles or free insulin was measured. The TEER of Caco-2 monolayer cells was measured again after removal of the nanoparticles or free insulin. The measurement apparatus was Millicell®-Electrical Resistance System.

FIG. 6 shows curves of TEER versus time. In the figure, the abscissa is time and the ordinate is change rate of TEER at specific time points. As shown in the figure, Nanoparticles 1 and Nanoparticles 2 resulted in rapid decreases of the TEER of Caco-2 monolayer cells to 50% and 54% of the initial values, respectively, within 2 hours after the start of the experiment; whereas the free insulin reduced the TEER of Caco-2 monolayer cells to about 85% of the initial value. Thus, in comparison with the free insulin, the insulin-loaded nanoparticles caused a significantly faster decrease of TEER of Caco-2 monolayer cells, indicating that the insulin-loaded nanoparticles were more likely able to open the tight junctions of cells. After 2 hours after the start of the experiment, the nanoparticles or the insulin solution were removed and the TEER was slowly picked up. The experiment shows that the insulin-loaded nanoparticles of the present invention can reversibly open the tight junction of cells, and can enhanced paracellular transport of insulin.

2. Measurement of Accumulative Permeation and Apparent Permeation Coefficient of Insulin

At specific time points, 20 μL samples were taken out from the receiving chamber, the insulin concentrations were measured by ELISA, and the accumulative permeation and apparent permeation coefficient of insulin were calculated.

The apparent permeation coefficient of insulin was calculated by the following formula:

Papp(cm/s)=Q/A×c×t;

Q is the total amount of insulin permeated (ng), A is the area of diffusion of monolayer cells (cm²), c is the initial concentration of insulin in the cell culture chamber (ng/cm³), t is the total time of the experiment.

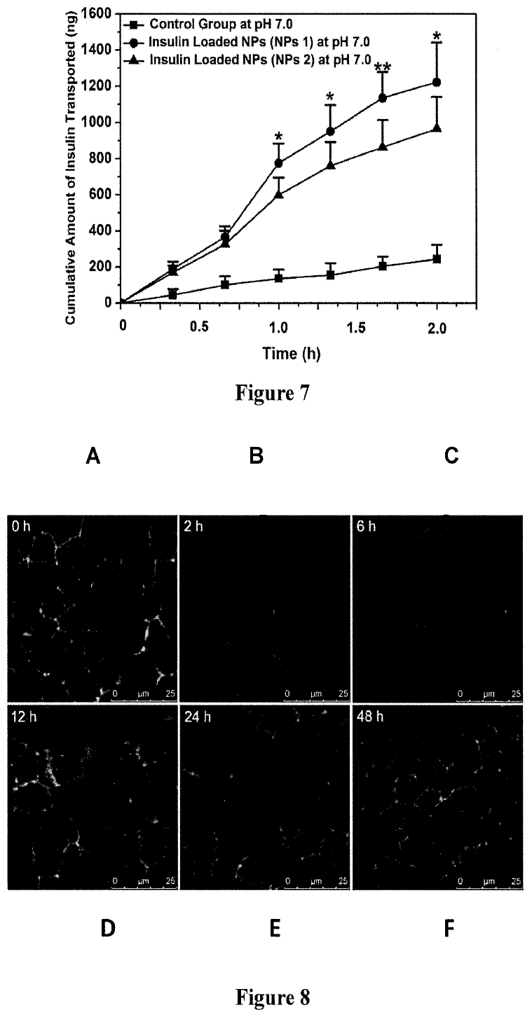

FIG. 7 is curves of accumulative amount of transported insulin versus time. As shown in the figure, in comparison with the free insulin, the insulin as loaded by the Nanoparticles 1 or Nanoparticles 2 showed significantly higher amount of transport.

The apparent permeation coefficients of insulin loaded by Nanoparticles 1 and Nanoparticles 2 were calculated to be 2.83±0.33×10⁻⁶ cm/s and 2.3±0.29×10⁻⁶ cm/s, respectively.

3. Observation of Changes in Tight Junctions of Cells

Caco-2 monolayer cells were fluorescent stained in the following manner: the cells were fixed with cold 4% paraformaldehyde solution for 15 min; the cells were washed with PBS; the cells were incubated for 30 min at room temperature with 5 μg/mL of primary antibody of tight junction protein; the cells were washed with PBS; the cells were incubated for 30 min at room temperature with 10 μg/mL of secondary antibody labeled with fluorescent reagent.

The morphology of the stained Caco-2 monolayer cells under the action of Nanoparticles 1 was observed by a confocal microscopy. After the action for 2 hours, Nanoparticles 1 were removed and the morphology of the cells was observed. The results are shown in FIG. 8.

FIG. 8 shows the morphologies of the cells before the action of the nanoparticles (FIG. 8A), under the action of Nanoparticle 1 (FIG. 8B) and after the nanoparticles were removed (FIG. 8C-F). It can be observed that tight junction proteins showed a continuous loop along cell boundary before the action of Nanoparticle 1. After two hours of the action of the nanoparticles, the tight junction proteins became blurred, and the loop along cell boundary became discontinuous, indicating that the tight junctions of cells were opened. When the nanoparticles were removed, the tight junction proteins became clear and the morphologies of proteins were gradually recovered. The above results indicated that, the insulin-loaded nanoparticles of the invention are capable of reversibly opening the tight junctions of cells.

### Example 8: Transcellular Transport of Insulin-Loaded Nanoparticles

Nanoparticles simultaneously labeled with FITC and Cy-5 were prepared using FITC-labeled chitosan and Cy-5 labeled insulin according to the steps of Example 1. The nanoparticles prepared at a flow rate of 25 mL/min had a particle size of 45 nm, which was named as Nanoparticles 3; the nanoparticles prepared at a flow rate of 1 mL/min had a particle size of 115 nm, which was named as Nanoparticles 4.

Transwell assay was performed using Caco-2 monolayer cells. 0.5 mL of medium (0.2 mg/mL, pH 7.0) containing Nanoparticles 3 or Nanoparticles 4 was added to a culture chamber, and the medium outside receiver was kept at pH 7.4. After incubation at 37° C. for 2 hours, the nanoparticles were removed, the cells were washed twice with a pre-warmed PBS solution and fixed with 4% paraformaldehyde, and the fixed cells were observed under a confocal microscopy. The free insulin labeled with Cy-5 was used for control experiment.

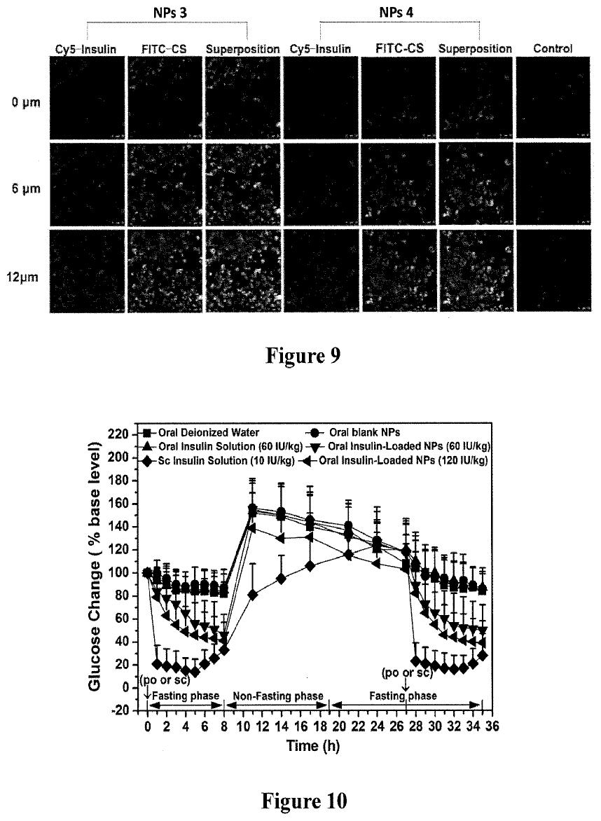

FIG. 9 shows confocal microscope photographs, in which Columns 1-3 show the results of characterization of Nanoparticles 3, Columns 4-6 show the results of the characterization of Nanoparticles 4, and Column 7 shows the results of characterization of the control group (free insulin). Nanoparticles 3 and Nanoparticles 4 had strong fluorescence signals of Cy-5 at depths of 6 μm and 12 μm after incubation for 2 hours, indicating that the insulin released from Nanoparticles 3 and Nanoparticles 4 was transported in Caco-2 monolayer cells. However, the control group had only weak Cy-5 fluorescence signals at depths of 6 μm and 12 μm. These results indicate that the nanoparticles of the present invention can enhance the insulin transport of cells.

### Example 9: Investigation of Hypoglycemic Effect of Insulin-Loaded Nanoparticles in Animals

The following animal experiments were approved by the Animal Protection and Use Center of Sun Yat-sen University. The experimental animals were provided by the Animal Experimental Center of Sun Yat-sen University.

Animals: Male SD rats weighing 220±20 g were given free access to water and feeding.

Establishment of type I diabetes mellitus model: a single injection of 70 mg/kg streptozotocin (in citrate buffer, 0.1 M, pH 4.2) into the abdominal cavity of rats was performed 2 weeks prior to the pharmacodynamic test. The rats with fasting blood-glucose concentration of 16.0 mmol/L or more were deemed as successful modeling.

The rats were grouped according to Table 6, subjected to measurement of basal values of blood glucose and administered separately.

TABLE 6

| | | Basal value |
|---|---|---|
| | | of blood |
| Group | Method and dose of administration | glucose |
| Group 1 | intragastrically administrated with insulin-loaded | 21.2 ± 3.8 mmol/L |
| | nanoparticles (Nanoparticles 1) at a dose of 60 | |
| | IU/kg | |
| Group 2 | intragastrically administrated with insulin-loaded | 20.5 ± 3.1 mmol/L |
| | nanoparticles (Nanoparticles 1) at a dose of 120 | |
| | IU/kg | |
| Group 3 | subcutaneously injected with a free insulin solution | 21.8 ± 2.8 mmol/L |
| | at a dose of 10 IU/kg | |
| Group 4 | intragastrically administrated with a free insulin | 22.3 ± 2.8 mmol/L |
| | solution at a dose of 60 IU/kg | |
| Group 5 | orally administrated with blank nanoparticles | 20.6 ± 3.1 mmol/L |
| Group 6 | orally administrated with deionized water | 21.5 ± 4.5 mmol/L |

The rats in the six groups were subjected to tail vein blood sampling at different time points, and the blood glucose levels were measured with a blood glucose meter. The rats were fasted but accessed to water before and during the experiment.

FIG. 10 shows curves of blood glucose level versus time in each of the groups of rats. As shown in the figure, the rats of Group 1 showed a blood glucose decreased by 51% within 8 hours after being intragastrically administrated with the nanoparticles at a dose of 60 IU/kg. The rats of Group 2 showed a blood glucose decreased by 59% within 8 hours after being intragastrically administrated with the nanoparticles at a dose of 120 IU/kg. The rats of Group 3 showed a sharp drop in blood glucose to 20% the basal level within 1 hour after being subcutaneously injected with the free insulin solution at a dose of 10 IU/kg, and this was further maintained for 4 hours. The rats of Group 4 showed no significant drop in blood glucose level after being orally administrated with the free insulin solution, while the rats of Group 5 and Group 6 showed similar results of blood glucose level. After 8 hours later, the rats were not fasted, and their blood glucose levels were picked up. On the next day, the same experiment was repeated, and similar results of blood glucose levels were observed.

The above results show that the insulin-loaded nanoparticles of the present invention can effectively reduce blood glucose level by oral administration, without causing a sharp decline in blood glucose level.

### Example 10: Intraperitoneal Glucose Tolerance Test

Samples to be Tested:

Hydroxypropylmethylcellulose phthalate (HPMCP) enteric-coated capsules comprising a lyophilized powder of Nanoparticles 1;

HPMCP enteric-coated capsules comprising a lyophilized powder of Nanoparticles 2;

HPMCP enteric-coated capsulescomprising a lyophilized powder of Nanoparticles 3 (average particle size of 240 nm, encapsulation efficiency 67%) prepared by the dropwise adding method;

HPMCP enteric-coated capsules containing insulin powder.

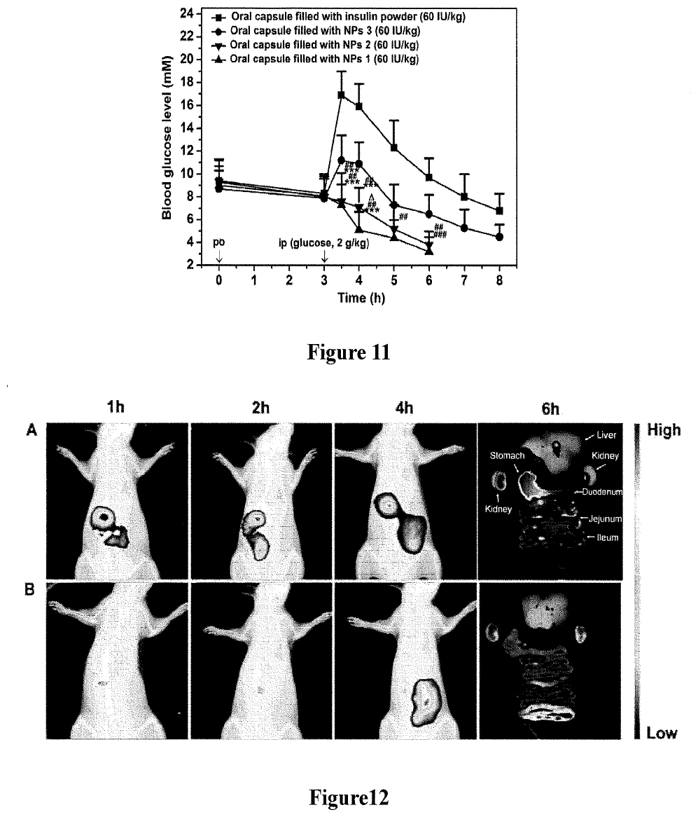

Experimental procedure: type I diabetic rats that were fasted for 12 hours were intragastrically administrated with capsules (60 IU/kg), and intraperitoneally injected with glucose solution (2 g/kg) after 3 hours. The blood glucose levels were measured and the results were shown in FIG. 11.

As shown in the figure, after being injected with the glucose solution, the mice administrated with the nanoparticles (Nanoparticles 1 or Nanoparticles 2) as prepared by the method of the present invention did not show an increase of blood glucose level; the mice administrated with the nanoparticles (Nanoparticles 3) as prepared by dropwise adding method showed an increase of blood glucose level of about 2 mM; while the mice administrated with free insulin showed an increase of blood glucose level of about 8 mM. The above results show that the insulin-loaded nanoparticles as prepared by the method of the present invention can effectively control the blood glucose level.

### Example 11: Biological Distribution of Insulin-Loaded Nanoparticles in Rats

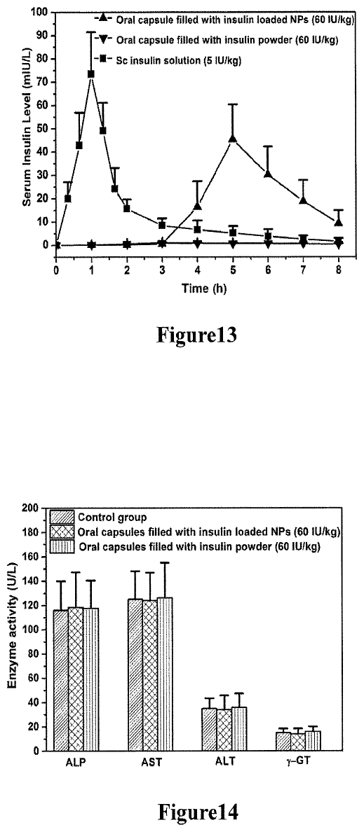

A suspension of Cy-7-labeled insulin-loaded nanoparticles was prepared using Cy-7-labeled insulin according to the method of Example 1, and then the suspension was lyophilized to prepare HPMCP capsules. The suspension and the capsules were intragastrically given to rats respectively, and in vivo distributions of insulin in rats were observed using a living body imaging technique. The results are shown in FIG. 12.

FIG. 12A shows pictures of 1 hour, 2 hours, 4 hours, 6 hours after intragastrical administration of the suspension; and FIG. 12B shows the pictures of 1 hour, 2 hours, 4 hours, and 6 hours after the intragastrical administration of the capsules. As shown in the figure, when 6 hours after the rats were administrated with the suspension, there was still a lot of insulin in stomachs of the rats, while some of insulin was located in liver, kidneys and intestines. When 6 hours after the rats were administrated with the capsules, most of insulin was located in intestines, while some of insulin was located in the livers and kidneys. The results show that the insulin-loaded nanoparticles encapsulated in the capsules could decrease the release of insulin in stomach, so that insulin was released more in small intestine, thereby enhancing the absorption of insulin via surface of small intestine and further increasing bioavailability thereof.

### Example 12: Test of In Vivo Pharmacokinetics

Type I diabetic rats were used in the test.

Group I: intragastrically administrated with HPMCP capsules of Nanoparticles 1 (60 IU/kg);

Group II: intragastrically administrated with HPMCP capsules of insulin powder (60 IU/kg);

Group III: subcutaneously injected with a free insulin solution (5 IU/kg).

Insulin concentration in serum was determined by porcine insulin ELISA kit. Relative bioavailability was calculated by comparing the area under the insulin level profile of the group of oral administration of capsules to the area under the drug-time curve of the group of subcutaneous injection.

FIG. 13 shows concentration-time curves of serum insulin n in rats. The concentration-time curve of Group I shows that after 3 hours from the administration, serum insulin began to be detected, and reached to a peak value after 5 hours (Cₘₐₓ=45.4 mIU/L). No insulin was detected in serum in the rats of Group II. The concentration-time curve of Group III shows that the insulin concentration in serum increased sharply after administration (which might cause a sharp drop of blood glucose), and reached to a peak value (Cₘₐₓ=73.5 mIU/L) after 1 hour from the administration. The relative bioavailability of the capsule comprising the insulin-loaded nanoparticles was calculated to be 10%.

### Example 13: Biosafety Evaluation

Rats were orally administrated with the capsules of Nanoparticles 1 and insulin capsules in 7 days, respectively. The control group was not administered. Using alkaline phosphatase, glutamic oxalacetic transaminase, glutamic-pyruvic transaminase, and glutamyl transpeptidase kits, the activity changes of corresponding enzymes in serum were measured. As shown in FIG. 14, in comparison with the rats administrated with free insulin and the rats of the control group, the rats administrated with Nanoparticle 1 showed no significant difference in various indexes. The results show that the insulin-loaded nanoparticles of the present invention have good biosafety.

Although specific embodiments of the present invention have been described in detail, those skilled in the art will appreciate that various modifications and variations of the details are possible in light of all of the teachings that have been disclosed and are within the scope of the present invention. The full scope of the invention is given by the appended claims and any equivalents thereof.

1. A method for preparing a therapeutic protein-loaded nanoparticle, comprising the steps as follows:
Step 1: providing a chitosan solution, a polyanion solution, a therapeutic protein solution and water;
Step 2: allowing the chitosan solution, the polyanion solution, the therapeutic protein solution and the water to pass through a first channel, a second channel, a third channel and a fourth channel, respectively, to reach a vortex mixing region, and mixing them;
wherein, the chitosan solution, the polyanion solution, the therapeutic protein solution and the water flow in channels at a constant flow rate; the flow rates of the chitosan solution, the polyanion solution, the therapeutic protein solution and the water are the same; and the flow rates of the chitosan solution, the polyanion solution, the therapeutic protein solution and the water are 1-120 mL/min(for example, 1-15 mL/min, 15-25 mL/min, 25-50 mL/min, 1-50 mL/min, 50-100 mL/min or 100-120 mL/min);
preferably, the therapeutic protein is insulin;
preferably, the polyanion is selected from the group consisting of sodium tripolyphosphate, alginic acid, heparin, hyaluronic acid, chondroitin sulfate, polyacrylic acid, polystyrenesulfonic acid; more preferably, the polyanion is sodium tripolyphosphate;
preferably, in Step 1, the concentration ratio (mg/mL:mg/mL:mg/mL) of the chitosan solution, the therapeutic protein solution and the polyanion solution is 1:0.1-0.7:0.2-0.5.

2. The method according to claim 1, wherein the concentration of the therapeutic protein solution in Step 1 is 0.1-0.7 mg/mL;
preferably, the pH of the therapeutic protein solution in Step 1 is 1.5-3.5;
preferably, the therapeutic protein solution in Step 1 further comprises hydrochloric acid;
preferably, the therapeutic protein solution in Step 1 is prepared by a method comprising the following step: dissolving the therapeutic protein in a hydrochloric acid solution with pH of 1.5-3.5.

3. The method according to claim 1 or 2, wherein the number-average molecular weight of chitosan in the chitosan solution in Step 1 is 10-500 KDa (for example, 10-50 KDa, 50-90 KDa, 90-150 KDa, 150-190 KDa, 190-250 KDa, 250-350 KDa or 350-500 KDa);
preferably, the pH of the chitosan solution in Step 1 is 5.0-6.0;
preferably, the chitosan solution in Step 1 is prepared by a method comprising the following steps: dissolving chitosan in an acetic acid solution with concentration of 0.1%-1% and adjusting the pH of the acetic acid solution to 5.0-6.0 using an alkali (for example, sodium hydroxide).

4. The method according to any one of claims 1-3, wherein the concentration of the polyanion solution in Step 1 is 0.2-0.5 mg/mL;
preferably, the polyanion solution in Step 1 further comprises a buffer agent, for example 4-hydroxyethylpiperazineethanesulfonic acid (HEPES);
preferably, the pH of the polyanion solution in Step 1 is 6.0-9.0;
preferably, the polyanion solution in Step 1 is prepared by a method comprising the following step: dissolving the polyanion in a HEPES buffer solution; more preferably, further comprising a step of adjusting the pH of the solution using an alkali (for example, sodium hydroxide).

5. The method according to any one of claims 1-4, wherein a suspension is obtained in Step 2, the suspension comprising the therapeutic protein-loaded nanoparticle;
preferably, the pH of the suspension obtained in Step 2 is 5.5-6.5 (for example, 5.5-5.8, 5.8-6.0, 6.0-6.2 or 6.2-6.5);
preferably, the method further comprises Step 3: lyophilizing the suspension;
preferably, the method further comprises adding a cryoprotectant to the suspension prior to Step 3;
preferably, the cryoprotectant is selected from mannitol and xylitol;
preferably, the cryoprotectant is a combination of mannitol and xylitol;
preferably, the ratio of the mass of mannitol, the mass of xylitol to the volume of the suspension is 0.2-0.5 g:0.5-1.5 g:100 mL.

6. The method according to any one of claims 1-5, wherein Step 2 is carried out in a multi-inlet vortex mixer, for example, a four-inlet vortex mixer;
preferably, the multi-inlet vortex mixer comprises a first member at the upper portion, a second member at the middle portion and a third member at the lower portion; the first member, the second member and the third member are cylinders having the same diameter; the first member is provided with a plurality of channels, the second member is provided with a vortex mixing region and a plurality of diversion regions, and the third member is provided with a passageway; the channels of the first member are in fluid communication with the diversion regions of the second member; the diversion regions are in fluid communication with the vortex mixing region in the second member; and the vortex mixing region of the second member is in fluid communication with the passageway of the third member;
preferably, the first member, the second member and the third member are hermetically connected with a threaded connection fitting;
preferably, the multi-inlet vortex mixer is made of a rigid material (for example, stainless steel).

7. A therapeutic protein-loaded nanoparticle, comprising a therapeutic protein, a chitosan and a polyanion, wherein the nanoparticle has a particle size of 30-240 nm (for example, 30-60 nm, 60-90 nm, 90-120 nm, 120-150 nm, 150-180 nm, 180-210 nm or 210-240 nm), the nanoparticle has a polydispersity index (PDI) of 0.13-0.19 (for example, 0.13-0.15, 0.15-0.17 or 0.17-0.19), and the nanoparticle has an encapsulation efficiency of not less than 65% (for example, not less than 65%, not less than 80% or not less than 90%);
preferably, the therapeutic protein is insulin;
preferably, the polyanion is selected from the group consisting of sodium tripolyphosphate, alginic acid, heparin, hyaluronic acid, chondroitin sulfate, polyacrylic acid, polystyrene sulfonic acid; more preferably, the polyanion is sodium tripolyphosphate;
preferably, the nanoparticle has a loading capacity of 10%-30%;
preferably, the nanoparticle has a Zeta potential of +5 mV to +15 mV;
preferably, the mass ratio of the chitosan and the polyanion in the nanoparticle is 1:0.2-0.35;
preferably, the mass ratio of the chitosan and the therapeutic protein in the nanoparticle is 1:0.1-0.7;
preferably, the nanoparticle exists in a suspension;
preferably, the nanoparticle is prepared by the method according to any one of claims 1-6.

8. A suspension, comprising the nanoparticle according to claim 7;
preferably, the suspension further comprises a cryoprotectant (for example, mannitol and/or xylitol);
preferably, the suspension is prepared by the method according to any one of claims 1-6.

9. A pharmaceutical composition, comprising the nanoparticle according to claim 7;
preferably, the pharmaceutical composition is useful in prevention or treatment of a disease that can be prevented or treated by the therapeutic protein comprised in the nanoparticle;
preferably, the therapeutic protein is insulin, and the pharmaceutical composition is useful in reducing blood glucose level in a subject;
preferably, the therapeutic protein is insulin, and the pharmaceutical composition is useful in prevention or treatment of hyperglycemia in a subject;
preferably, the hyperglycemia comprises stress-induced hyperglycemia, diabetes (including type I diabetes and type II diabetes) and impaired glucose tolerance;
preferably, the subject is a mammal, for example, a bovine, an equine, a goat, a porcine, a canine, a feline, a rodent, a primate; for example, the subject is a human.

10. A pharmaceutical preparation, comprising the nanoparticle according to claim 7, the suspension according to claim 8 or the pharmaceutical composition according to claim 9;
preferably, the pharmaceutical preparation further comprises a pharmaceutically acceptable excipient;
preferably, the pharmaceutical preparation is a lyophilized preparation;
preferably, the pharmaceutical preparation is a capsule;
preferably, the shell of the capsule is hydroxypropyl methylcellulose ester shell;
preferably, the pharmaceutical preparation is useful in prevention or treatment of a disease that can be prevented or treated by the therapeutic protein comprised in the nanoparticle;
preferably, the therapeutic protein is insulin, and the pharmaceutical preparation is useful in reducing blood glucose level in a subject;
preferably, the therapeutic protein is insulin, and the pharmaceutical preparation is useful in prevention or treatment of hyperglycemia in a subject;
preferably, the hyperglycemia comprises stress-induced hyperglycemia, diabetes (including type I diabetes and type II diabetes) and impaired glucose tolerance;
preferably, the subject is a mammal, for example, a bovine, an equine, a goat, a porcine, a canine, a feline, a rodent, a primate; for example, the subject is a human.

11. Use of the nanoparticle according to claim 7 in manufacture of a pharmaceutical composition, wherein the pharmaceutical composition is useful in prevention or treatment of a disease that can be prevented or treated by the therapeutic protein comprised in the nanoparticle;
preferably, the therapeutic protein is insulin, and the disease is hyperglycemia;
preferably, the hyperglycemia comprises stress-induced hyperglycemia, diabetes (including type I diabetes and type II diabetes) and impaired glucose tolerance;
preferably, the subject is a mammal, for example, a bovine, an equine, a goat, a porcine, a canine, a feline, a rodent, a primate; for example, the subject is a human.

12. A method for preventing or treating a disease, comprising administering to a subject in need thereof the nanoparticle according to claim 7, the suspension according to claim 8, the pharmaceutical composition according to claim 9 or the pharmaceutical preparation according to claim 10, wherein the disease is a disease that can be prevented or treated by the therapeutic protein comprised in the nanoparticle, the suspension, the pharmaceutical composition or the pharmaceutical preparation;
preferably, the therapeutic protein is insulin, and the disease is hyperglycemia;
preferably, the hyperglycemia comprises stress-induced hyperglycemia, diabetes (including type I diabetes and type II diabetes) and impaired glucose tolerance;
preferably, the subject is a mammal, for example, a bovine, an equine, a goat, a porcine, a canine, a feline, a rodent, a primate; for example, the subject is a human.
